(19) United States
(12) Patent Application Publication
(10) Pub. No.: US 2009/0012355 A1
(43) Pub. Date: Jan. 8, 2009

(54) Condom Ring with Multiple Vibration Modes

(76) Inventor: Chien-Feng LIN, Jhonghe City (TW)

Correspondence Address: TROXELL LAW OFFICE PLLC, 5205 LEESBURG PIKE, SUITE 1404, FALLS CHURCH, VA, 22041, US

(21) Appl. No.: 12/167,847

(22) Filed: Jul. 3, 2008

(30) Foreign Application Priority Data
Jul. 5, 2007 (TW) 096210918

(51) Int. Cl. A61F 5/00; A61H 1/00

(52) U.S. Cl. 600/41; 600/38; 601/46

(57) ABSTRACT

A vibrating condom ring includes a motive element, a power supply element, multiple conduction elements, a switch element, and a control element. The switch element controls the operation of the control element; the control element is electrically connected to the motive element and the power supply element respectively through the conductive element; and the control element is capable of controlling the vibrating frequency and model for the user may control the vibrating frequency and model of the vibrating condom ring by means of the switch element to increase sex pleasure.

### BACKGROUND OF THE INVENTION

(a) Field of the Invention

The present invention is related to a condom ring with multiple vibration modes, and more particularly, to a power ring to be inserted onto a condom for increasing sexual funs while encouraging the use of the condom to effectively prevent spreading of social disease and AIDS.

(b) Description of the Prior Art

Statistics shows that most of AIDS patients are in the age groups of the young and the prime of life in the world, wherein one fifth are falling in the age group of 15˜20. In Taiwan, the number of those who affected by AIDS by end of September 2006 rises up to 3422; among them, approximately 90% had infection through sex, and approximately 40% fall within the age group of 20˜29, indicating that the attitude about sex of most of the young people knowing only to indulging in pursuit of short while of sexual pleasure and satisfaction fail to develop correct and mature recognition at a time when the sex becomes more permissive and knowledge about sex is popularly available. Therefore, Centers for Disease Control of Department of Health, Taiwan launched propaganda programs to teach the younger people to always use a condom before making love; however, the results are not particularly good.

As illustrated in FIGS. 1, 2, and 3 of the accompanying drawings, a vibrating condom ring generally available in the market is disposed with a body 1; a ring 11 and a vibrator 12 are disposed to the body 1; and the vibrator is further comprised of a batter 121, a switch 122, a first conductive plate 123, a second conductive plate 124, and a motor 125. The first conductive plate 123 is disposed at where between an electrode of the battery 121 and the motor 125; the second conductive plate 124 is separately coupled to another electrode of the batter 121 and to an outer casing of the motor 125; and the switch 122 is coupled to the second conductive plate 124 to control whether powerwil be supplied from the battery 121.

When put in use, the body 1 is inserted onto a condom by means of the ring 11; the switch 122 is pressed for the second conductive plate 124 to contact the outer casing of the motor 125, and for the battery 122 to supply power to and start the motor 125 and further for the body 1 to produce vibration effect. However, the vibrating condom ring of the prior art provides only one vibration at fixed frequency or one vibration mode, e.g., a single vibrating speed and that prevents readjustment depending on the individual user.

### SUMMARY OF THE INVENTION

The primary purpose of the present invention is to provide a vibrating condom ring to enjoy more fun in sex, significantly increase use of condom, and effectively prevent social diseases and AIDS from spreading.

To achieve the purpose, a vibrating condom ring of the present invention is comprised of a ring and a vibrating component. The vibrating component includes a motive element, a power supply element, multiple conduction elements, a switch element, and a control element. The switch element controls the operation of the control element; the control element is electrically connected to the motive element and the power supply element respectively through the conductive element; and the control element is capable of controlling the vibrating frequency and model. Accordingly, the user may control the vibrating frequency and model of the vibrating condom ring by means of the switch element to increase sex pleasure.

### BRIEF DESCRIPTION OF THE DRAWINGS

FIG. 1 is a perspective view of a vibrating condom ring of the prior art.

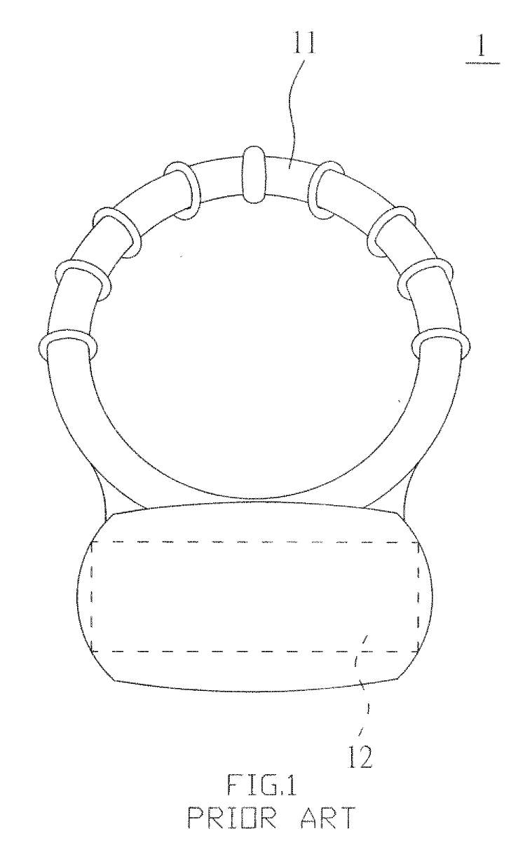

FIG. 2 is a schematic view showing a construction of the prior art.

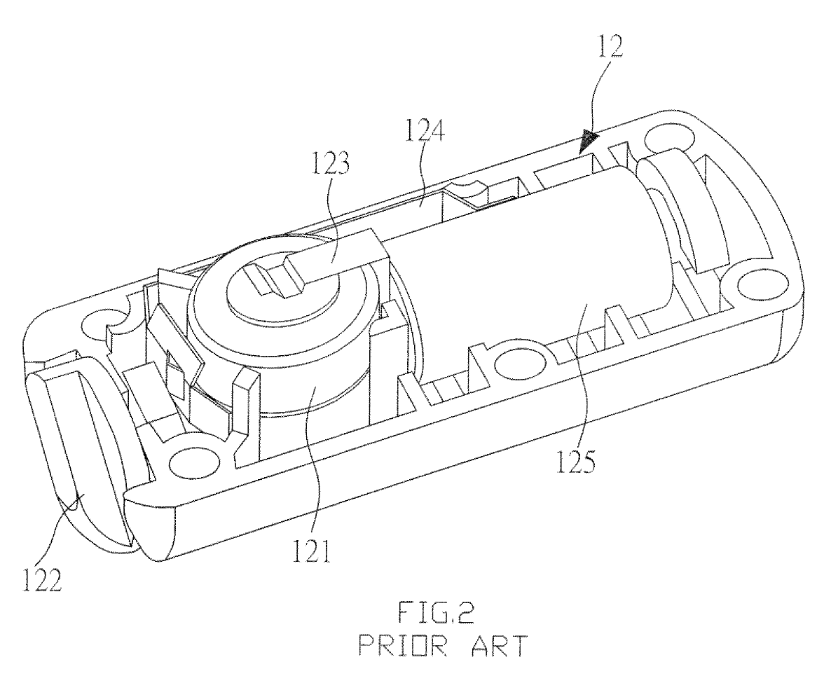

FIG. 3 is a block chart of the prior art.

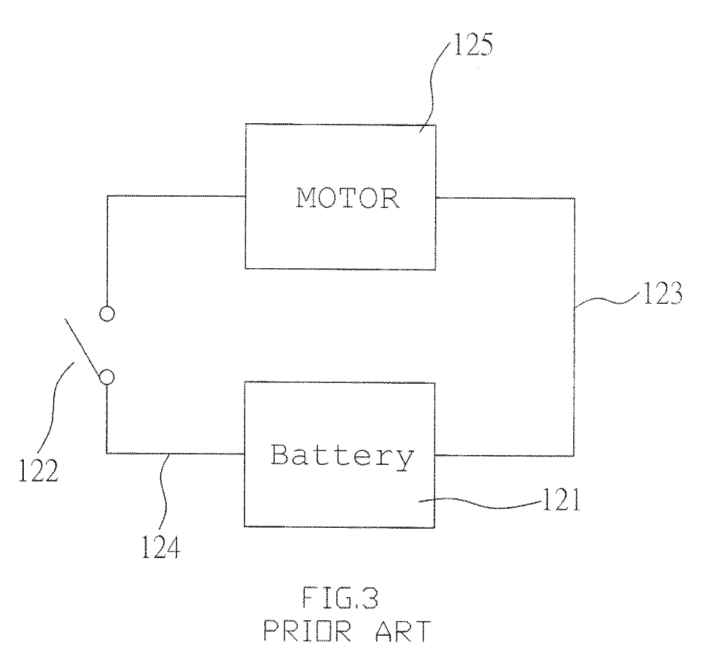

FIG. 4 is an exploded view showing a construction of a vibrating condom ring in the present invention.

FIG. 5 is an exploded view showing a vibrating component of the present invention.

FIG. 6 is a schematic view showing a construction of the present invention.

FIG. 7 is a block chart showing the vibrating component in the present invention.

FIG. 8 is an exploded view showing another construction of the present invention.

### DETAILED DESCRIPTION OF THE PREFERRED EMBODIMENTS

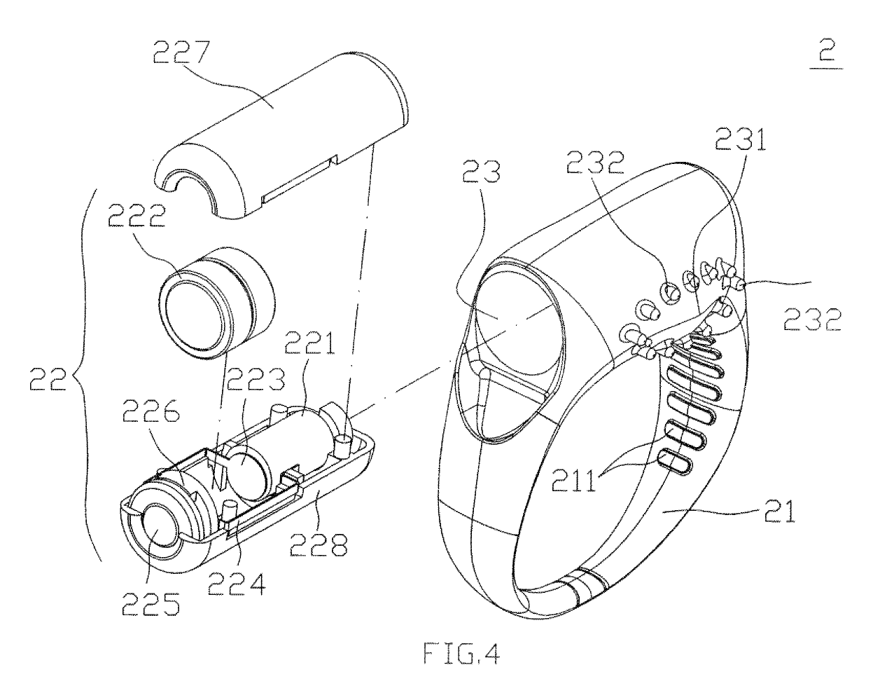

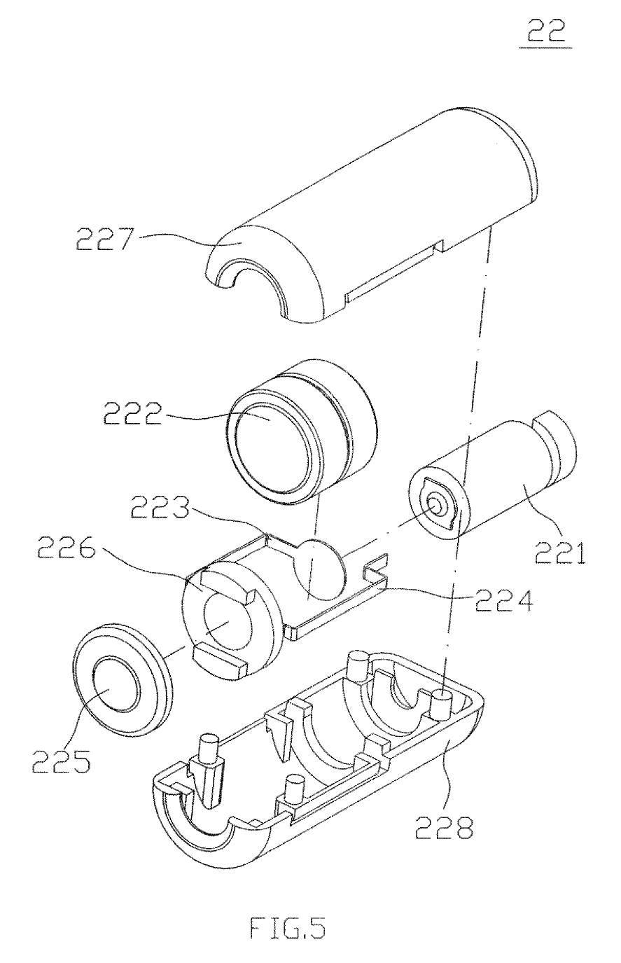

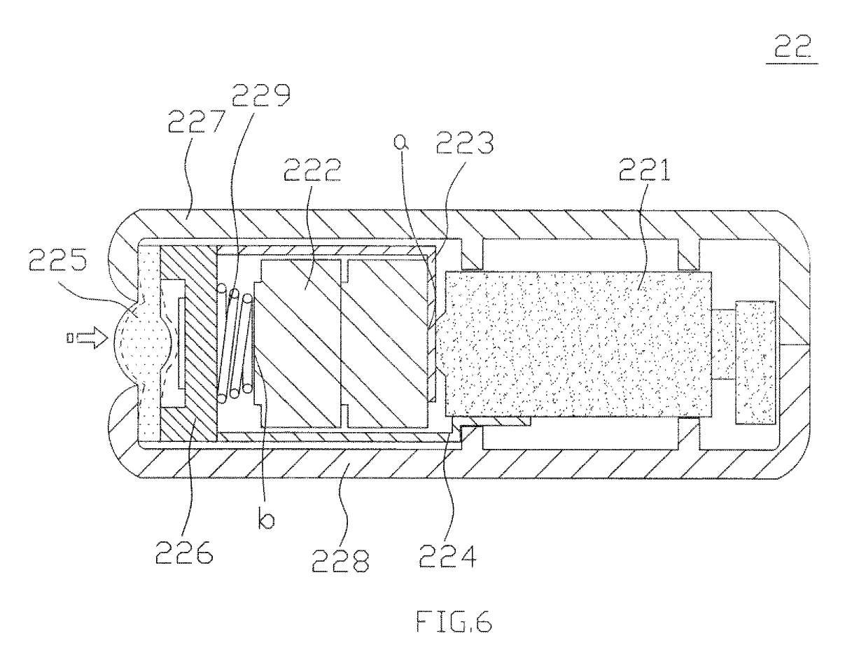

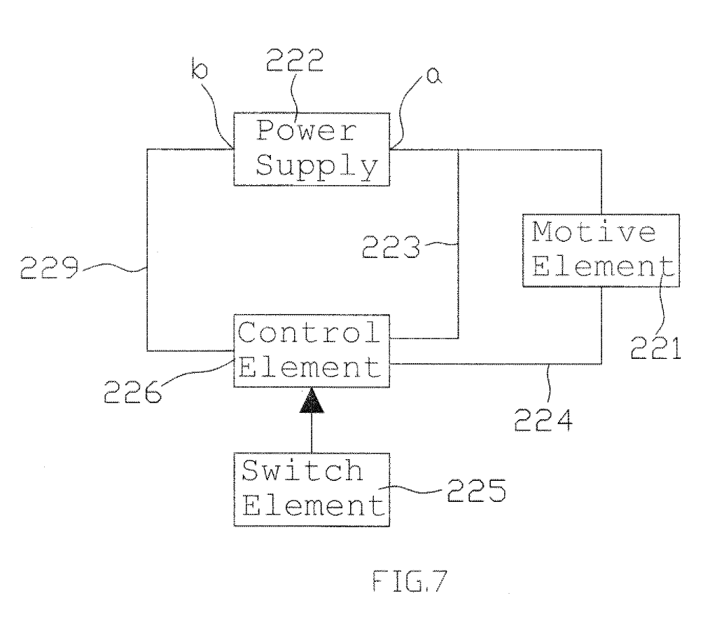

Referring to FIGS. 4 through 7, a vibrating condom ring 2 of the present invention is comprised of an insertion ring 21 and a vibrating component 22. The vibrating component 22 is disposed with a motive element 221 (e.g., a vibrating motor), a power supply element 222 (e.g., a battery) and a conductive member. The conductive member is comprised of a first conductive element 223, a second conductive element 224, and a third conductive element 229 (e.g., conductive plate or spring). The power supply element 222 is electrically connected to motive element 221 by means of the first conductive element 223, the second conductive element 224 respectively to operate the motive element 221 and the condom ring 2 to vibrate.

A switch element 225 and a control element 226 are disposed to the vibrating component 22. The switch element 225 controls operation of the control element 226 and is related to an electronic key switch. The first conductive element 223 constitutes electric connection among the control element 226, one electrode a in the power supply element 222, and the motive element 221; and the third conductive element 229 constitutes electric connection between another electrode b of the power supply element 222 and the control element 226. The control element 226 transmits different signals to control the motive element 221 to create various vibration frequencies and modes while allowing the vibration frequency and mode adjustable to continuous or intermittent vibration at low, middle, or high speed.

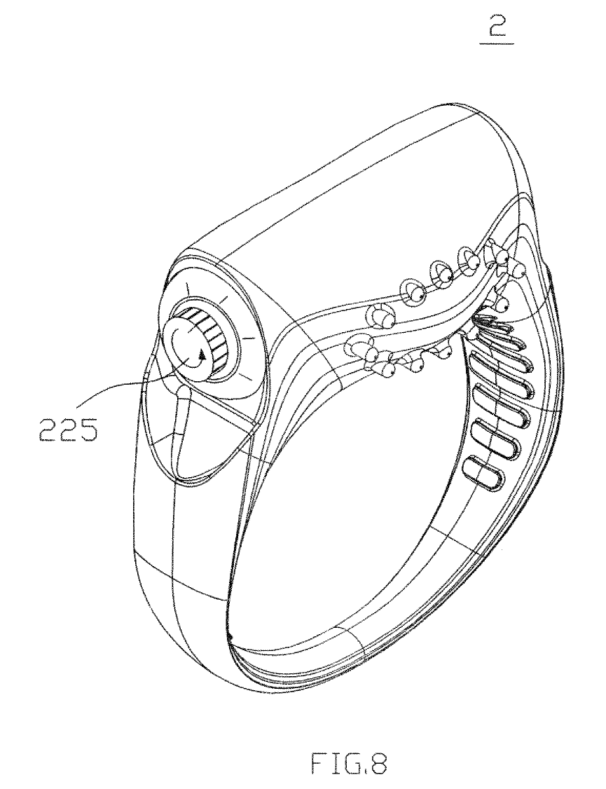

In use, the condom ring 2 is inserted onto a condom by means of the insertion ring 21 and the user starts the control element 226 by operating the switch element 225 for the control element 226 to transmit signals to operate the motive element 221. In a preferred embodiment of the present invention, a first, a second, a third, and a fourth vibration modes are made available to the control element 226, respectively, continuous vibration at middle speed, continuous vibration at high speed, intermittent vibration at high speed (i.e., vibration at high speed spaced at interval, and stop. Therefore, when the user presses the switch element 225 for the first time, the motive element 221 operates the first vibration mode, (i.e., continuous vibration at middle speed); presses for the second time, the second vibration mode (continuous vibration at high speed); presses for the third time, the second vibration mode (intermittent vibration at high speed); and presses for the fourth time, the vibration stops. Alternatively, six vibration modes are provided, respectively, continuous vibration at middle speed, stop, continuous vibration at high speed, stop, intermittent vibration at high speed, and stop. Certainly, the switch element 22 is made in a form a of a turning knob as illustrated in FIG. 8.

It is to be noted that the present invention is capable of controlling through the control element the vibrating condom ring to deliver different vibrating frequency and mode for enjoying more the sex, significantly increasing use of condom, and thus to effectively present social diseases and AIDS from spreading.

Furthermore, on the distal end to the insertion ring 21 of the vibrating condom ring 2 is disposed with a chamber 23 as illustrated in FIG. 4 to receive the vibrating component 22; and the vibrating component 22 is further provided with an upper casing 227 and a lower casing 228 with both of the upper and the lower casings 227, 228 to cover up on each other. All the parts contained in the vibrating component 22 are mounted in a space defined at where between both of the upper and the lower casings 227, 228. Accordingly, those parts are accessible for repair or replacement by separating both of the upper and the lower casings 227, 228 from the vibrating condom ring 2 to replace with a new power supply element for maintaining normal power supply.

A protruding portion 231 is formed on an outer side of the chamber 23 and multiple nipples 232 are disposed to the outer peripheral of the protruding portion 231 for those nipples 232 and the protruding portion 231 to massage the vagina while having sex. Multiple ribs 211 are disposed on an inner circumference of the insertion ring 21 to improve packing strength between the vibrating condom ring 2 and the condom for prevent the vibrating condom ring 2 from sliding.

The prevent invention provides an improved structure of a vibrating condom ring with multiple vibrating frequency and modes, and the application for a utility patent is duly filed accordingly. However, it is to be noted that the preferred embodiments disclosed in the specification and the accompanying drawings are not limiting the present invention; and that any construction, installation, or characteristics that is same or similar to that of the present invention should fall within the scope of the purposes and claims of the present invention

I claim,

1. A vibrating condom ring provided with multiple vibration modes with the vibrating condom ring containing is comprised of a vibrating condom ring and a vibrating component; the vibrating component comprising a motive element; a power supply element connected to the motive element by means of a conductive member; a control element controlling vibrating frequency and mode of the motive element; and a switch element controlling operation of the control element; wherein the conductive member containing a first conductive element, a second conductive element, and a third conductive element; an electrode of the first conductive element constituting electrical connection among the control element, an electrode of the power supply element, and the motive element; the second conductive element constituting electric connection between the control element and the motive element; and the third conductive element constituting electric connection between another electrode of the power supply element and the control element.

2. The vibrating condom ring provided with multiple vibration modes as claimed in claim 1, wherein the vibrating condom ring is further comprised of a chamber to receive the vibrating component.

3. The vibrating condom ring provided with multiple vibration modes as claimed in claim 2, wherein the vibrating component is further comprised of an upper casing and a lower casing capable of covering up onto each other; and all the parts contained in the vibrating component are mounted to a space defined where between the upper and the lower casings.

4. The vibrating condom ring provided with multiple vibration modes as claimed in claim 2, wherein a protruding portion is formed on an outer side of the chamber.

5. The vibrating condom ring provided with multiple vibration modes as claimed in claim 4, wherein multiple nipples are disposed to an outer peripheral of the protruding portion.

6. The vibrating condom ring provided with multiple vibration modes as claimed in claim 1, wherein multiple ribs are disposed on an inner circumference of the insertion ring.

7. The vibrating condom ring provided with multiple vibration modes as claimed in claim 1, wherein the vibrating frequency and mode includes multiple vibration modes comprised of any combination among continuous vibration and intermittent vibration respectively at low, middle, and high speeds as desired.

8. The vibrating condom ring provided with multiple vibration modes as claimed in claim 1, wherein the switch element is related to an electronic key switch or a turning knob.
